(19) United States
(12) Patent Application Publication
(10) Pub. No.: US 2024/0252662 A1
(43) Pub. Date: Aug. 1, 2024

(54) PEPTIDE DRUG CONJUGATES SPECIFIC TO FIBRONECTIN ISOTYPES FOR CANCER THERAPY

(71) Applicant: Case Western Reserve University, Cleveland, OH (US)

(72) Inventors: Zheng-Rong LU, Cleveland, OH (US); Songqi GAO, Cleveland, OH (US)

(21) Appl. No.: 18/561,384

(22) PCT Filed: May 20, 2022
(86) PCT No.: PCT/US2022/030172

§ 371 Date: Nov. 16, 2023

Related U.S. Application Data
(60) Provisional application No. 63/191,078, filed on May 20, 2021.

(51) Int. Cl. A61K 47/64; A61K 38/00; A61P 35/00; C07K 7/06

(52) U.S. Cl. CPC A61K 47/64; A61P 35/00; C07K 7/06; A61K 38/00

(57) ABSTRACT

An anticancer peptide conjugate is described that comprises the following formula: P-L-A wherein: P is a peptide that includes an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or variants thereof in which one or more L-amino acids have been replace with a corresponding D-amino acid; A is an antitumor agent; and L is an optional linker that covalently links the peptide to the antitumor agent, and pharmaceutically acceptable salts thereof. Methods of using the anticancer peptide conjugates to treat cancer are also described.

### CROSS-REFERENCE TO RELATED APPLICATIONS

This application claims priority to U.S. Provisional Application No. 63/191,078, filed on May 20, 2021, which is hereby incorporated by reference in its entirety.

### GOVERNMENT FUNDING

This invention was made with government support under grant No. CA211762 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety.

### BACKGROUND

Prostate cancer (PCa) is the second most common cause of cancer mortality in men. There are about 200,000 new prostate cancer cases diagnosed in the USA each year. Most prostate cancer patients are diagnosed with low-grade lesions and will not die from the disease. Prostate cancer patients are treated with surgery, radiation therapy, hormone therapy, chemotherapy, immunotherapy, and targeted therapy, based on the stage of the disease. Litwin et al., JAMA 317(24) 2532-2542 (2017). Unfortunately, some of the patients diagnosed with high-risk prostate cancer would develop recurrent disease, including castrate-resistant and hormone-refractory prostate cancer, and distant metastasis, and do not respond to available therapies. Virgo et al., J Clin Oncol 39(11), 1274-1305 (2021). Consequently, about 34,000 patients are prematurely died of prostate cancer each year.

Although various chemotherapeutics, targeted therapy and radiopharmaceuticals have been used to treat high-risk PCa, their poor pharmacokinetics, biodistribution, and tumor accumulation often result in poor therapeutic outcomes. Various drug delivery systems, including nanoparticles, have been tested for delivery of anticancer therapeutics for treating PCa. However, these delivery systems are unable to provide efficient drug delivery in solid tumors due to the barrier of dense extracellular matrix (ECM). Therefore, these delivery systems have not achieved the expected therapeutic efficacy to cure cancer in clinical application.

EDB-FN is an oncofetal subtype of fibronectin. Its overexpression is associated with angiogenesis, cancer cell epithelial-to-mesenchymal transition (EMT), invasion, metastasis, and drug resistance. Kaspar et al., Int J Cancer 118(6) 1331-9 (2006). Clinical data have shown EDB-FN is highly expressed in the ECM of in aggressive human carcinomas, including PCa, but little in normal tissues. Vaidya et al., Cells, 9(8), 1826 (2020). Dense ECM presents a formidable barrier for conventional nanosized drug delivery systems. Z. R. Lu, P. Qiao, Mol Pharm 15(9) 3603-3616 (2018). However, EDB-FN is a promising ECM oncotarget for specific drug delivery to treat high-risk PCa.

Previously, the inventors have developed a small peptide of seven amino acids named ZD2 (TVRTSAD) to target EDB fragment. See U.S. Pat. No. 10,124,073. A targeted MRI contrast agent ZD2-N3-Gd(HP-DO3A) (MT218) has been developed using the peptide for accurate detection of high-risk PCa. See International Patent No. WO2021/247967. The abundant EDB-FN in tumor ECM is readily accessible to the small peptide targeted contrast agent to allow robust signal enhancement throughout aggressive PCa at low doses, indicating high efficiency of the peptide to deliver imaging agents in solid tumors. Currently, the targeted contrast agent has been approved by the FDA for clinical development for PCa diagnosis.

JQ1 is a potent BET bromodomain protein inhibitor. JQ1 and its derivatives have been tested in preclinical and clinical studies for treating a variety of cancers, including prostate cancer. Welti et al., Clin Cancer Res 24(13), 3149-3162 (2018). JQ1 has been shown to effectively treat castration-resistant prostate cancer. However, these new drugs have shown significant poor pharmacokinetics and dose-dependent toxic side-effects. In addition, a recent study showed that JQ1 is effective to treat PCa, but may induce cancer cell EMT and metastasis with some PCa cell types and tumor models. Wang et al., J Clin Invest 130(4), 1782-1792 (2020). The inventors have not observed such side effects in EDB-FN positive, invasive PCa.

There remains an unmet clinical need of novel diagnostics and therapeutics to for precision diagnosis and efficacious treatment in clinical management of high-risk PCa.

### SUMMARY

The inventors have developed novel peptide drug conjugates for specific and efficacious treatment of high-risk prostate cancer (PCa), including recurrent and metastatic PCa. They hypothesized that ZD2 peptide drug conjugates can also efficiently deliver anti-cancer therapeutics into high-risk, EDN-FN positive PCa in high concentration, similar as MT218, to achieve for efficacious treatment under image-guidance with MRI and MT218.

The inventors have designed, synthesized, and characterized ZD2 peptide anticancer drug conjugates targeting EDB-FN for efficient delivery of the drugs throughout high-risk PCa. Design and development of small peptide drug conjugates targeting an ECM oncoprotein have the potential overcome this barrier. EDB-FN is an ECM protein and is highly accessible for tumor targeting as compared to the biomarkers expressed on cancer cell surface. As shown in the MR images of EDB-FN rich PCa tumors in rodent models, ZD2 targeted MT218 results in robust signal enhancement throughout the tumors, indicating its ability to overcome the dense ECM by targeting the ECM oncoprotein to achieve highly efficient delivery of diagnostics into solid tumors. It is expected that ZD2 peptide will also efficiently deliver anti-cancer drug into the solid tumors to achieve efficacious cancer therapy.

### BRIEF DESCRIPTION OF THE FIGURES

FIG. 1 provides graphs showing the cell cytotoxicity of JAAP in PC3 and C4-2 prostate cancer, MDA-MB-231 and 4T1 breast cancer, DLD-1 and DLD-1-DR drug resistant colon cancer, Panc-1 and BXPC3 pancreatic cancer cells, cal-27, SCC4 and HSC3 head and neck cancer cells.

FIG. 2 provides graphs showing the cell viability of different cancer cells treated with ZD2-HZ-JAAP.

FIG. 3 provides a graph and image showing the Antitumor efficacy of ZD2-HZ-JAAP in treating MDA-MB-231 breast cancer. Top row tumors are from the mice treated with ZD2-HZ-JAAP and the bottom tumors with PBS.

FIG. 4 provides a graph showing the body weight of the mice bearing MDA-MB-231 breast cancer treated with ZD2-HZ-JAAP.

FIG. 5 provides a graph showing antitumor efficacy of ZD2-HZ-JAAP in treating PC3 prostate cancer.

FIG. 6 provides a graph showing the body weight of the mice bearing PC3 prostate cancer treated with ZD2-HZ-JAAP.

FIG. 7 provides a graph and image showing the antitumor efficacy of ZD2-HZ-JAAP in treating 4T1 breast cancer. Top tumors are from the mice treated with ZD2-HZ-JAAP and the bottom tumors with PBS.

FIG. 8 provides a graph showing the body weight of the mice bearing 4T1 breast cancer treated with ZD2-HZ-JAAP.

FIG. 9 provides a graph showing the cell viability of MDA-MB-231 breast cancer cells treated with ZD2-Ams-JAAP, ZD2-Am-JAAP, JQ1.

FIG. 10 provides graphs showing the tumor volumes and body weight of the mice bearing MDA-MB-231 xenografts treated with ZD2-PEG-HZ-JAAP and PBS.

FIG. 11 provides graphs showing the cell viability of different cancer cells treated with ZD2-Am-DOX and free DOX.

FIG. 12 provides graphs showing the viability of different cancer cells treated with ZD2-PEG-HZ-DOX.

FIG. 13 provide a graph showing the 4T1 tumor growth curves for PBS, free DOX and ZD2-HZ-DOX treated groups.

FIG. 14 provides a graph showing the average bodyweight curves for PBS, free DOX, and ZD2-HZ-DOX treated groups.

FIG. 15 provides a graph showing the mouse survival curves for PBS, free DOX and ZD2-HZ-DOX treated groups.

### DETAILED DESCRIPTION

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

### Definitions

As used in the description of the invention and the appended claims, the singular forms “a,” “an,” and “the” are intended to include the plural forms as well, unless the context clearly indicates otherwise. In addition, the recitations of numerical ranges by endpoints include all numbers subsumed within that range (e.g., 1 to 5 includes 1, 1.5, 2, 2.75, 3, 3.80, 4, 5, etc.).

The term “specific binding affinity” as used herein, refers to the capacity of a compound (e.g., a peptide or anticancer peptide conjugate) to be taken up by, retained by, or bound to a particular biological component to a greater degree than other components. Compounds that have this property are said to be “targeted” to the “target” component. The anticancer peptide conjugates and methods described herein relate to targeting fibronectin, fibronectin complexes, and oncofetal fibronectin. Anticancer agents that lack this property are said to be “non-specific” or “non-targeted” agents.

“Treating”, as used herein, means ameliorating the effects of, or delaying, halting or reversing the progress of a disease or disorder. The word encompasses reducing the severity of a symptom of a disease or disorder and/or the frequency of a symptom of a disease or disorder.

The language “effective amount” or “therapeutically effective amount” refers to a nontoxic but sufficient amount of the composition used in the practice of the invention that is effective to stimulate endothelial cell growth at the site of nanoparticle delivery. The desired treatment may be prophylactic and/or therapeutic. That result can be reduction and/or alleviation of the signs, symptoms, or causes of a disease or disorder, or any other desired alteration of abiological system. An appropriate therapeutic amount in any individual case may be determined by one of ordinary skill in the art using routine experimentation.

A “subject”, as used therein, can be a human or non-human animal. Non-human animals include, for example, livestock and pets, such as ovine, bovine, porcine, canine, feline and murine mammals, as well as reptiles, birds and fish. Preferably, the subject is human.

“Pharmaceutically acceptable carrier” refers herein to a composition suitable for delivering an active pharmaceutical ingredient, such as the composition of the present invention, to a subject without excessive toxicity or other complications while maintaining the biological activity of the active pharmaceutical ingredient. Protein-stabilizing excipients, such as mannitol, sucrose, glucose, polysorbate-80 and phosphate buffers, polymers such as polyethylene glycol (PEG), polyvinyl alcohol (PVA), pluronics, are typically found in such carriers, although the carriers should not be construed as being limited only to these compounds.

The terms “peptide(s)”, “protein(s)” and “polypeptide(s)” are used interchangeably herein. As used herein, “polypeptide” refers to any peptide or protein comprising two or more amino acids joined to each other by peptide bonds or modified peptide bonds (i.e., peptide isomers). “Polypeptide(s)” refers to both short chains, commonly referred as peptides, oligopeptides or oligomers, and to longer chains generally referred to as proteins.

The term “fragment” refers to any subject peptide having an amino acid residue sequence shorter than that of a polypeptide whose amino acid residue sequence is shown herein. Fragments lack one or more amino acids from one or both ends of the peptide. For example, a fragment can include 1, 2, 3, 4, 5, 6, 7, 8, 9, 10, or more than 10 fewer amino acids than the non-fragmented peptide.

The following abbreviations for amino acids are used throughout the application: A=Ala=Alanine, T=Thr=Threonine, V=Val=Valine, C=Cys=Cysteine, L=Leu=Leucine, Y=Tyr=Tyrosine, I=Ile=Isoleucine, N=Asn=Asparagine, P=Pro=Proline, Q=Gln=Glutamine, F=Phe=Phenylalanine, D=Asp=Aspartic Acid, W=Trp=Tryptophan, E=Glu=Glutamic Acid, M=Met=Methionine, K=Lys=Lysine, G=Gly=Glycine, R=Arg=Arginine, S=Ser=Serine, H=His=Histidine.

As used herein, the term “organic group” is used for the purpose of this invention to mean a hydrocarbon group that is classified as an aliphatic group, cyclic group, or combination of aliphatic and cyclic groups (e.g., alkaryl and aralkyl groups). In the context of the present invention, suitable organic groups for protein disulfide isomerase inhibitors are those that do not interfere with the compounds anticancer activity. In the context of the present invention, the term “aliphatic group” means a saturated or unsaturated linear or branched hydrocarbon group. This term is used to encompass alkyl, alkenyl, and alkynyl groups, for example.

As used herein, the terms “alkyl”, “alkenyl”, and the prefix “alk-” are inclusive of straight chain groups and branched chain groups and cyclic groups, e.g., cycloalkyl and cycloalkenyl. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Lower alkyl groups are those including at most 6 carbon atoms. Examples of alkyl groups include haloalkyl groups and hydroxyalkyl groups.

Unless otherwise specified, “alkylene” and “alkenylene” are the divalent forms of the “alkyl” and “alkenyl” groups defined above. The terms, “alkylenyl” and “alkenylenyl” are used when “alkylene” and “alkenylene”, respectively, are substituted. For example, an arylalkylenyl group comprises an alkylene moiety to which an aryl group is attached.

Unless otherwise indicated, the term “heteroatom” refers to the atoms O, S, or N. The term “heteroaliphatic” includes branched and unbranched aliphatic groups that contain at least one heteroatom (e.g., O, S, N). In some embodiments, the term “heteroaliphatic” includes a aliphatic that contains 2 to 12 carbon atoms, 1 to 3 rings, 1 to 4 heteroatoms, and O, S, and/or N as the heteroatoms.

### Anticancer Peptide Conjugates

In one aspect, the present invention provides an anticancer peptide conjugate comprising the following formula: P-L-A wherein: P is a fibronectin-binding peptide; A is an antitumor agent; and L is an optional linker that covalently links the peptide to the antitumor agent, and pharmaceutically acceptable salts thereof.

The anticancer peptide conjugates described herein include targeting peptides with a peptide sequence that specifically binds to and/or complexes with fibronectin. Fibronectins are structural glycoproteins that form insoluble extracellular matrix via complexation with other extracellular matrix components, including collagen and fibrin. Oncofetal fibronectin, which has an extradomain A (EDA), B (EDB) or type III domain, is a cancer-related isoform of fibronectin expressed in malignant tumors. Clinical evidence indicates that the oncofetal fibronectin in the extracellular matrix plays a biological role in the aggressiveness of cancer cells.

The fibronectin-binding peptides are a chain of amino acids that is about 3 to about 20 amino acids in length. In some embodiments, the targeting peptides have a size from 4 to 15 amino acids, while in further embodiments the targeting peptides have a size from 5 to 10 amino acids, or 6 to 8 amino acids.

Anticancer peptide conjugates including the fibronectin-binding peptides can be administered systemically to a subject, such as by intravenous or parenteral administration, and readily target fibronectin (e.g., extradomain B fibronectin; EDB-FN and/or extradomain A fibronectin; EDA-FN) to target the anticancer compound to tumor tissue.

In some embodiments, the fibronectin-binding peptides are unmodified peptides. In other embodiments, the fibronectin-binding peptides can be subject to various changes, substitutions, insertions, and deletions to provide modified peptides, where such changes provide for certain advantages in its use. In this regard, targeting peptides that bind to and/or complex with EDB-FN and/or EDA-FN can be substantially homologous with, rather than be identical to, the sequence of a recited peptide where one or more changes are made and it retains the ability to function as specifically binding to and/or complexing with EDB-FN and/or EDA-FN.

In some embodiments, analogs of fibronectin peptide binding proteins can be used. The term “analog” includes any peptide having an amino acid residue sequence substantially identical to a sequence specifically shown herein in which one or more residues have been conservatively substituted with a functionally similar residue and that specifically binds to and/or complexes with EDB-FN and/or EDA-FN as described herein. Examples of conservative substitutions include the substitution of one non-polar (hydrophobic) residue, such as isoleucine, valine, leucine or methionine for another, the substitution of one polar (hydrophilic) residue for another, such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such peptide displays the requisite binding activity.

In some modified fibronectin-binding peptides, chemical derivatives of the fibronectin binding peptides are used. “Chemical derivative” refers to a subject peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-benzylhistidine. Also included as chemical derivatives are those polypeptides, which contain one or more naturally occurring amino acid derivatives of the twenty standard amino acids.

Examples of chemical derivatives of amino acids that can be included in a chemical derivative of a fibronectin binding peptide include: 4-hydroxyproline may be substituted for proline; 5-hydroxylysine may be substituted for lysine; 3-methylhistidine may be substituted for histidine; homoserine may be substituted for serine; and ornithine may be substituted for lysine. Peptides described herein also include any peptide having one or more additions and/or deletions or residues relative to the sequence of a peptide whose sequence is shown herein, so long as the requisite binding specificity or activity is maintained.

In some embodiments, the fibronectin-binding peptide comprises or consists of an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or variants thereof in which one or more L-amino acids have been replace with a corresponding D-amino acid. In a further embodiment, the targeting peptide comprises or consists of the amino acid sequence SEQ ID NO: 1.

TABLE 1
Fibronectin-binding Peptides

| Sequence | |
|---|---|
| Identification | |
| Number | Amino Acid Sequence |
| SEQ ID NO: 1 | Thr-Val-Arg-Ser-Ala-Asp |
| SEQ ID NO: 2 | Asn-Trp-Gly-Asp-Arg-Ile-Leu |
| SEQ ID NO: 3 | Asn-Trp-Gly-Lys-Pro-Ile-Lys |
| SEQ ID NO: 4 | Ser-Gly-Val-Lys-Ser-Ala-Phe |
| SEQ ID NO: 5 | Gly-Val-Lys-Ser-Tyr-Asn-Glu |
| SEQ ID NO: 6 | Ile-Gly-Lys-Thr-Asn-Thr-Leu |
| SEQ ID NO: 7 | Ile-Gly-Asn-Ser-Asn-Thr-Leu |
| SEQ ID NO: 8 | Ile-Gly-Asn-Thr-Ile-Pro-Val |
| SEQ ID NO: 9 | Leu-Tyr-Ala-Asn-Ser-Pro-Phe |

The inventors have determined that replacing one or more L-amino acid of the fibronectin-binding proteins can improve the stability of the fibronectin binding protein against metabolic breakdown. Systematic substitution of one or more amino acids of a consensus sequence with D-amino acid of the same type (e.g., an enantiomer; D-lysine in place of L-lysine) may be used to generate more stable polypeptides. Thus, a fibronectin binding protein may be all L-, all D-, or mixed D, L polypeptides. The amino acid sequences of the polypeptides with D-amino acids are identical to the sequences of the polypeptides to which they correspond, except for the presence of the one or more corresponding D-amino acid residue(s). In particular, replacing Serine and/or Arginine has been shown to improved stability. Methods of preparing peptides including D-amino acids are known to those skilled in the art. See U.S. Patent Publication 2020/0283817. Accordingly, in some embodiments, one or more of the amino acids of the fibronectin-binding peptide can be replaced with one or more corresponding D-amino acids. For example, in some embodiments, P is a fibronectin-binding peptide that includes an amino acid sequence selected from the group consisting of SEQ ID NO: 10, SEQ ID NO: 11, SEQ ID NO: 12, SEQ ID NO: 13, and SEQ ID NO: 14, as shown in Table 2.

TABLE 2
Higher Stability Fibronectin-binding Peptides

| Sequence | |
|---|---|
| Identification | |
| Number | Amino Acid Sequence |
| SEQ ID NO: 10 | Thr-Val-D-Arg-D-Ser-Ala-Asp |
| SEQ ID NO: 11 | Asn-Trp-Gly-Asp-D-Arg-Ile-Leu |
| SEQ ID NO: 12 | D-Ser-Gly-Val-Lys-D-Ser-Ala-Phe |
| SEQ ID NO: 13 | Gly-Val-Lys-D-Ser-Tyr-Asn-Glu |
| SEQ ID NO: 14 | Ile-Gly-Asn-D-Ser-Asn-Thr-Leu |
| SEQ ID NO: 15 | Leu-Tyr-Ala-Asn-D-Ser-Pro-Phe |

The fibronectin-binding peptides can be synthesized by any of the techniques that are known to those skilled in the polypeptide art, including recombinant DNA techniques. Synthetic chemistry techniques, such as a solid-phase Merrifield-type synthesis, can be used for reasons of purity, antigenic specificity, freedom from undesired side products, ease of production and the like. A summary of the many techniques available can be found in Steward et al., “Solid Phase Peptide Synthesis”, W. H. Freeman Co., San Francisco, 1969; Bodanszky, et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv. Enzymol., 32:221-96, 1969; Fields et al., int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis, each of which is incorporated herein by reference. Appropriate protective groups usable in such synthesis are described in the above texts and in J. F. W. McOmie, “Protective Groups in Organic Chemistry”, Plenum Press, New York, 1973, which is incorporated herein by reference.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

Using a solid phase synthesis as an example, a protected or derivatized amino acid can be attached to an inert solid support through its unprotected carboxyl or amino group. The protecting group of the amino or carboxyl group can then be selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group can then be removed from this newly added amino acid residue, and the next amino acid (suitably protected) is then added, and so forth. After all the desired amino acids have been linked in the proper sequence, any remaining terminal and side group protecting groups (and solid support) can be removed sequentially or concurrently, to afford the final linear polypeptide.

The anticancer peptide conjugate also includes an optional linker L that covalently links the peptide to the antitumor agent. Additional residues may be added at either terminus of a fibronectin-binding peptide for the purpose of providing a “linker” by which the fibronectin-binding peptide can be conveniently linked and/or affixed to the anticancer agent. Linkers can include amino acid residue linkers, or non-peptide linkers. Furthermore, in some embodiments, the anticancer agent is bonded directly to the fibrobectin-binding peptide. For example, in some embodiments, the anticancer agent JQ-1 is directly bonded to the fibronectin-binding peptide.

In some embodiments, the antitumor agent is directly attached to a functional group on the linker or fibronectin-binding peptide capable of reacting with the antitumor agent. For example, lysines that have a free amino group that can be capable of reacting with a carbonyl-containing group, such as an anhydride or an acid halide, or with an alkyl group containing a good leaving group (e.g., a halide). Peptides can also contain glutamic and aspartic acids. The carboxylate groups of these amino acids also present attractive targets for functionalization using carbodiimide activated linker molecules; cysteines can also be present which facilitate chemical coupling via thiol-selective chemistry (e.g., maleimide-activated compounds).

Amino acid residue linkers are usually at least one residue and can be 40 or more residues, more often 1 to 10 residues. Typical amino acid residues used for linking are glycine, tyrosine, cysteine, lysine, glutamic and aspartic acid, or the like. In addition, a subject targeting peptide agent can differ by the sequence being modified by terminal-NH₂ acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half-life of the polypeptides in solutions, particularly biological fluids where proteases may be present. In this regard, polypeptide cyclization is also a useful terminal modification, and is particularly preferred also because of the stable structures formed by cyclization and in view of the biological activities observed for such cyclic peptides as described herein.

In some embodiments, the linker is a non-peptide linker. In further embodiments, the non-peptide linker is a non-peptide aliphatic or heteroaliphatic linker. In yet further embodiments, the non-peptide linker is covalently attached to the antitumor agent through an amide, hydrazone, ester, or disulfide bond. In some embodiments, the non-peptide linker is covalently attached to the antitumor agent through an amide or hydrazone bond.

The non-peptide linker can include a first portion that is about 1 to about 10 atoms in lengths and second portion that acts as a spacer. The portion of the linker that acts a spacer can include a non-peptide polymer that includes but is not limited to a polyalkyleneoxide, polyvinyl alcohol, polyethylene glycol (PEG), polypropylene glycol (PPG), co-poly (ethylene/propylene) glycol, polyoxyethylene (POE), polyurethane, polyphosphazene, polysaccharides, dextran, polyvinylpyrrolidones, polyvinyl ethyl ether, polyacryl amide, polyacrylate, polycyanoacrylates, lipid polymers, chitins, hyaluronic acid, and heparin. For more detailed descriptions of spacers for non-peptide linkers, see, for example, WO/2006/107124, which is incorporated by reference herein. Typically such linkers will have a range of molecular weight of from about 1 kDa to 50 kDa, depending upon a particular linker. For example, a typical PEG has a molecular weight of about 1 to 5 kDa, and polyethylene glycol has a molecular weight of about 5 kDa to 50 kDa, and more preferably about 10 kDa to 40 kDa.

The anticancer peptide conjugate also includes an antitumor agent (A). A wide variety of antitumor agents are known to those skilled in the art. Examples of antitumor agents include include angiogenesis inhibitors such as angiostatin K1-3, DL-α-difluoromethyl-ornithine, endostatin, fumagillin, genistein, minocycline, staurosporine, and (±)-thalidomide; DNA intercalating or cross-linking agents such as bleomycin, carboplatin, carmustine, chlorambucil, cyclophosphamide, cisplatin, phenanthriplatin, melphalan, mitoxantrone, and oxaliplatin; DNA synthesis inhibitors such as methotrexate, 3-Amino-1,2,4-benzotriazine 1,4-dioxide, aminopterin, cytosine P-D-arabinofuranoside, 5-Fluoro-5′-deoxyuridine, 5-Fluorouracil, gaciclovir, hydroxyurea, and mitomycin C; DNA-RNA transcription regulators such as actinomycin D, daunorubicin, doxorubicin, homoharringtonine, and idarubicin; enzyme inhibitors such as S(+)-camptothecin, curcumin, (−)-deguelin, 5,6-dichlorobenz-imidazole 1-β-D-ribofuranoside, etoposine, formestane, fostriecin, hispidin, cyclocreatine, mevinolin, trichostatin A, tyrophostin AG 34, and tyrophostin AG 879, Gene Regulating agents such as 5-aza-2′-deoxycitidine, 5-azacytidine, cholecalciferol, 4-hydroxytamoxifen, melatonin, mifepristone, raloxifene, all trans-retinal, all trans retinoic acid, 9-cis-retinoic acid, retinol, tamoxifen, and troglitazone; Microtubule Inhibitors such as colchicine, dolostatin 15, nocodazole, paclitaxel, podophyllotoxin, rhizoxin, vinblastine, vincristine, vindesine, and vinorelbine; and various other antitumor agents such as 17-(allylamino)-17-demethoxygeldanamycin, 4-Amino-1,8-naphthalimide, apigenin, brefeldin A, cimetidine, dichloromethylene-diphosphonic acid, leuprolide, luteinizing-hormone-releasing hormone, pifithrin-α, rapamycin, thapsigargin, and bikunin, and derivatives thereof.

The specific antitumor agents chosen can be those known to be more effective against the type of cancer being targeted. For example, antitumor agents suitable for treating prostate cancer include BET bromodomain inhibitors, Cabazitaxel, Degarelix, Taxotere (Docetaxel), Enzalutamide, Jevtana (Cabazitaxel), Lupron or Viadur (Leuprolide Acetate), Prednisone, Prolia or Xgeva (Denosumab), Provenge (Sipuleucel-T), Xofigo (Radium 223 Dichloride), Sipuleucel-T, Xtandi (Enzalutamide), and Zytiga (Abiraterone Acetate).

In some embodiments, the antitumor agent is a BET bromodomain inhibitor. BET bromodomain is a useful strategy to target c-Myc. Delmore et al., Cell, 146(6):904-17 (2011). Examples of BET bromodomain inhibitors include JQ1, JQ1 derivatives, iBET, PLX51107, and JAAP. Filippakopoulos et al., 468:1067-1073 (2010); Erkes et al., Pigment Cell Melanoma Res., 32(5):687-696 (2019). Other BET bromodomain inhibitors include UMB-32 derivatives, Bromodomain inhibitor-8 derivatives, CPI-0610 derivatives, I-BET762 derivatives, RVX derivatives and OTX015 derivatives. In the context of the present invention, suitable organic groups for BET bromodomain inhibitor derivatives are those that do not interfere with the compounds inhibitory activity. In In some embodiments, the BET bromodomain inhibitor is JQ1 or JAAP.

A number of specific anticancer peptide conjugates have been prepared by the inventions. Accordingly, in some embodiments, the conjugate is selected from the group consisting of ZD2-HZ-JQ1, ZD2-JQ-1132, ZD2-JQ-1245, ZD2-PEG-JQ1-AAP, ZD2-doxorubicin conjugated with a hydrazone, ZD2-doxorubicin conjugated with an amide, and ZD2-PEG-DOX. The structures and preparation of these compounds are described in the Examples provided herein.

### Cancer Treatment

Another aspect of the present invention provides a method of treating cancer in a subject. The method includes administering a therapeutically effective amount of an anticancer peptide conjugate to a subject in need thereof, the anticancer peptide conjugate comprising the following formula: P-L-A, wherein: P is a fibronectin-binding peptide; A is an antitumor agent; and L is an optional linker that covalently links the peptide to the antitumor agent, and pharmaceutically acceptable salts thereof. In some embodiments, P is a peptide that includes an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or variants thereof in which one or more L-amino acids have been replace with a corresponding D-amino acid. In some embodiments, the amino acid sequence of the anticancer peptide conjugate is SEQ ID NO: 1.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma. In some embodiments, the cancer is breast cancer, oral cancer, pancreatic cancer, or prostate cancer.

In some embodiments, the present invention provides a method of treating prostate cancer. Prostate cancer is cancer found in the prostate, which is an exocrine gland of the male reproductive system, and exists directly under the bladder, in front of the rectum. Most prostate cancers are adenocarcinomas. Prostate cancer, as used herein, includes precancer forms such as prostatic intraepithelial neoplasia. The severity of prostate cancer is generally evaluated using a Gleason score, with range from 2 to 10, obtained by adding the score for a predominant pattern to a secondary pattern, with pattern scores ranging from 1 to 5 and increasing score numbers indicating a more advanced and/or aggressive form of prostate cancer. For example, a Gleason score of 6 or more can indicate the presence of a worse than average, or severe, form of prostate cancer. In some embodiments of the invention, the subject has one or more symptoms of prostate cancer. Symptoms of prostate cancer include trouble urinating, decreased force in the stream of urine, blood in the urine, blood in the semen, general pain in the lower back, hips or thighs, discomfort in the pelvic area, bone pain, and erectile dysfunction. Additional screening and diagnostic tests can be performed to help determine if a subject has prostate cancer. Screening tests include a digital rectal exam, and a test for higher-than-normal levels of prostate-specific antigen, which diagnostic tests include a transrectal ultrasound or a biopsy of prostate tissue which is evaluated for the presence of prostate cancer cells.

In some embodiments, the cancer being treated is drug-resistant cancer. Cancer can become drug resistant through a variety of different mechanisms. Vasan et al., Nature, 575, 299-309 (2019). Tumor size plays an important role in cancer's resistance to drugs. Other factors include tumor heterogeneity (e.g., oncogenic driver mutations), the formation of physical barriers, and immune evasion resulting from changes in the tumor microenvironment. Cancer that has become drug resistant can be identified by those skilled in the art.

The anticancer peptide conjugate used in the method of cancer treatment can include any of the anticancer peptide conjugates described herein. In some embodiments, the non-peptide linker of the anticancer peptide conjugate is a non-peptide aliphatic or heteroaliphatic linker. In further embodiments, the non-peptide linker of the anticancer peptide conjugate is covalently linked to the antitumor agent through an amide or hydrazone bond. In additional embodiments, the antitumor agent of the anticancer peptide conjugate is a BET bromodomain inhibitor (e.g., JQ1 or JAAP). In some embodiments, P of the anticancer peptide conjugate is a peptide that includes an amino acid sequence selected from the group consisting of SEQ ID NO: 10, SEQ ID NO: 11, SEQ ID NO: 12, SEQ ID NO: 13, and SEQ ID NO: 14. In yet further embodiments, the anticancer peptide conjugate is selected from the group consisting of ZD2-HZ-JQ1, ZD2-JQ-1132, ZD2-JQ-1245, ZD2-PEG-JQ1-AAP, ZD2-doxorubicin conjugated with a hydrazone, ZD2-doxorubicin conjugated with an amide, and ZD2-PEG-DOX.

In some embodiments, additional treatment of the cancer is provided. For example, additional types of treatment for prostate cancer are surgery (e.g., radical prostatectomy, pelvic lymphadenectomy, transurethral resection of the prostate, orchiectomy, or cryosurgery), radiation therapy (e.g., internal radiation therapy using strontium-89, proton beam radiation therapy) hormone therapy, biologic therapy, targeted therapy (e.g., monoclonal antibody therapy), and high-intensity focused ultrasound. When additional treatment is provided, the anticancer peptide conjugates can be administered to the subject prior to, during, or post use of the additional treatment method.

### Formulation and Administration

Formulation of the anticancer peptide conjugate to be administered will vary according to the route of administration selected (e.g., solution, emulsion, capsule, and the like). In some embodiments, the anticancer peptide conjugate is administered together with a pharmaceutically acceptable carrier. Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like.

The preparation of a pharmacological composition that contains active ingredients dissolved or dispersed therein is well understood in the art. Typically, such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

Any anticancer peptide conjugate may also be used in the form of a pharmaceutically acceptable salt. Acids, which are capable of forming salts with the polypeptides, include inorganic acids such as trifluoroacetic acid (TFA) hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like.

Bases capable of forming salts with the polypeptides include inorganic bases, such as sodium hydroxide, ammonium hydroxide, potassium hydroxide and the like; and organic bases such as mono-, di- and tri alkyl and aryl-amines (e.g., triethylamine, diisopropylamine, methylamine, dimethylamine and the like) and optionally substituted ethanolamines (e.g., ethanolamine, diethanolamine and the like).

Alternately, if the anticancer peptide conjugates possess carboxylic acid groups, these groups can be converted to pharmaceutically acceptable esters using techniques known in the art. Alternatively, if an ester is present on the compound, the ester can be converted to a pharmaceutically acceptable ester using transesterification techniques.

The following examples are included for purposes of illustration and are not intended to limit the scope of the invention.

### EXAMPLES

### Example 1: Data for ZD2 JQ1 and JAAP Conjugates

### 1. Synthesis and Characterization of JAAP

JQ1-carboxylic acid, 4-aminoacetophenone, DCC (N,N-dicyclohexylcarbodiimide), and DMAP in a ratio of 1:1.5:1.5:1.5 were dissolved in DCM (dichloromethane). The solution was stirred at room temperature (r.t.) overnight. After filtration to remove DCU, the product was purified by preparative HPLC, and lyophilized to give a yellow powder. The Yield is 40%. The product was characterized by ESI mass spectrometry and H-NMR. (M+1) m/z, 518 observed; 518.13 calculated for C₂₇H₂₄ClN₅O₂S. The product was also analyzed by HPLC, and the UV spectrum was measured.

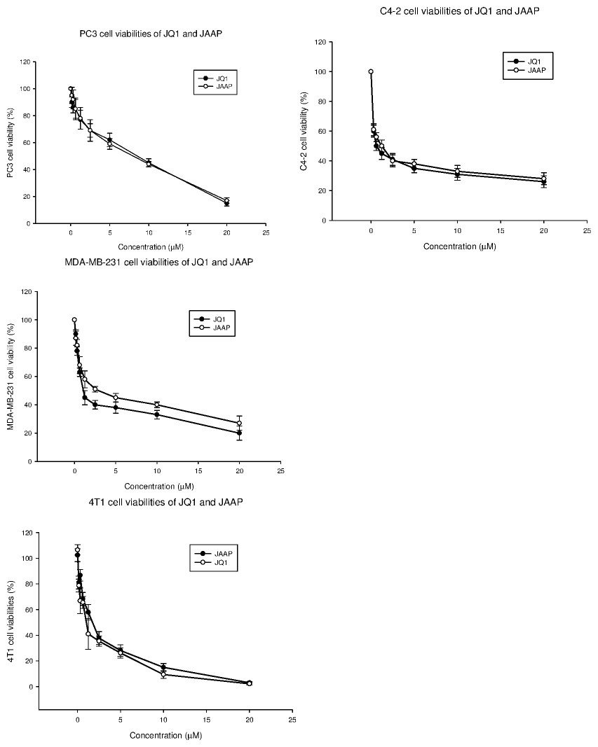

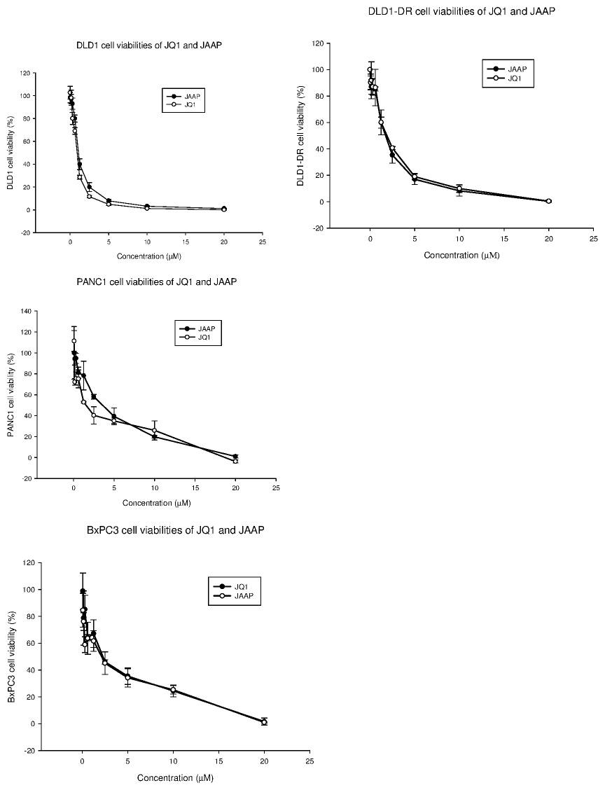

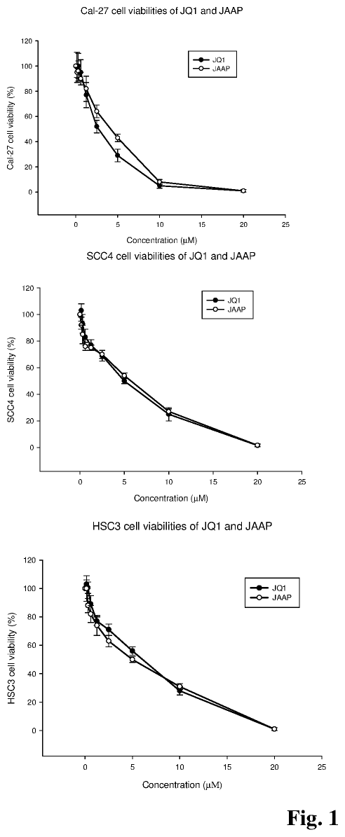

The cell cytotoxicity of JAAP was tested using PC3 and C4-2 prostate cancer, MDA-MB-231 and 4T1 breast cancer, DLD-1 and DLD-1-DR drug resistant colon cancer, Panc-1 and BXPC3 pancreatic cancer cells, cal-27, SCC4 and HSC3 head and neck cancer cells, and demonstrated that the cytotoxicity of JAAP is similar as JQ1. See FIG. 1.

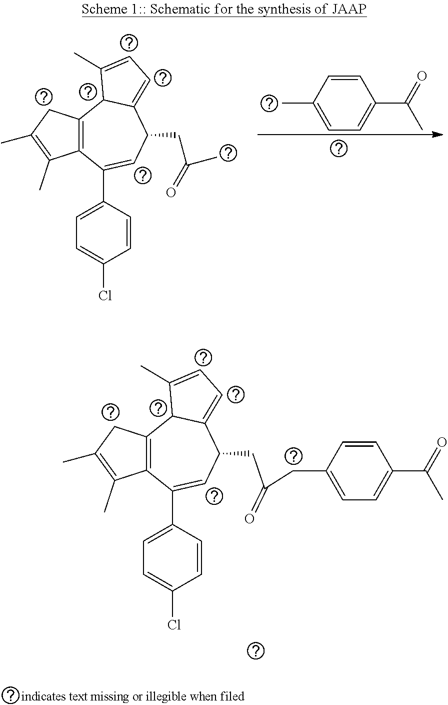

2. Synthesis and Characterization of ZD2-HZ-JAAP with a Hydrazone Bond

ZD2 peptide was synthesized using standard solid-phase chemistry. Then succinic anhydride was conjugated to N-terminus of ZD2 peptide on the resin. After that, t-butyl carbazate was reacted with the peptide in the presence of an excess of HBTU. The product was subsequently cleaved from the resin using a cocktail of TFA/water/TIBS (96.5/2.5/1). This hydrazine ZD2-NH—NH₂ (50 mg) was reacted with JAAP (20 mg) in DMF 4 ml containing (100 mg) molecular sieve to form the ZD2-HZ-JAAP conjugate with a hydrazone bond product, ZD2-HZ-JAAP. The product was precipitated in ethyl ether, washed by water (yield 50%), and characterized by ESI mass spectrometry. (M+1) m/z, 1363 observed; 1362.54 calculated for C₆₀H₈₀ClN₁₇O₁₆S.

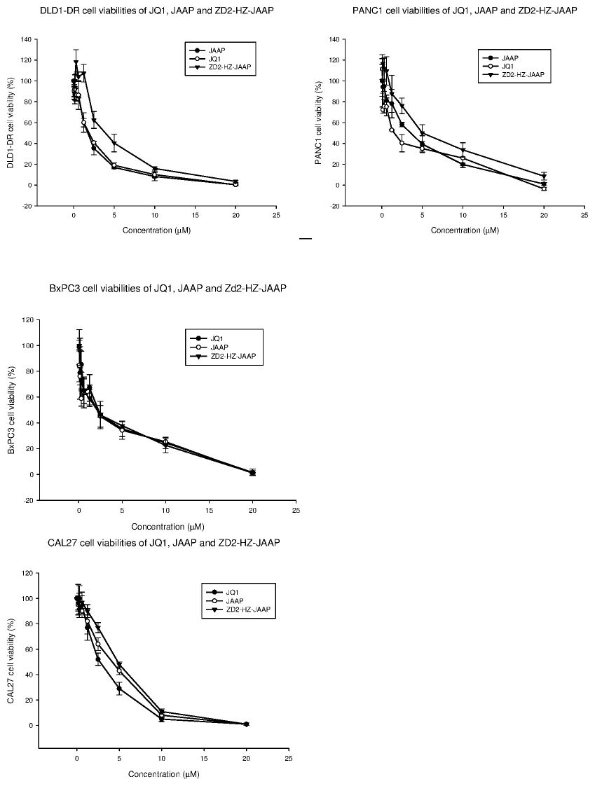

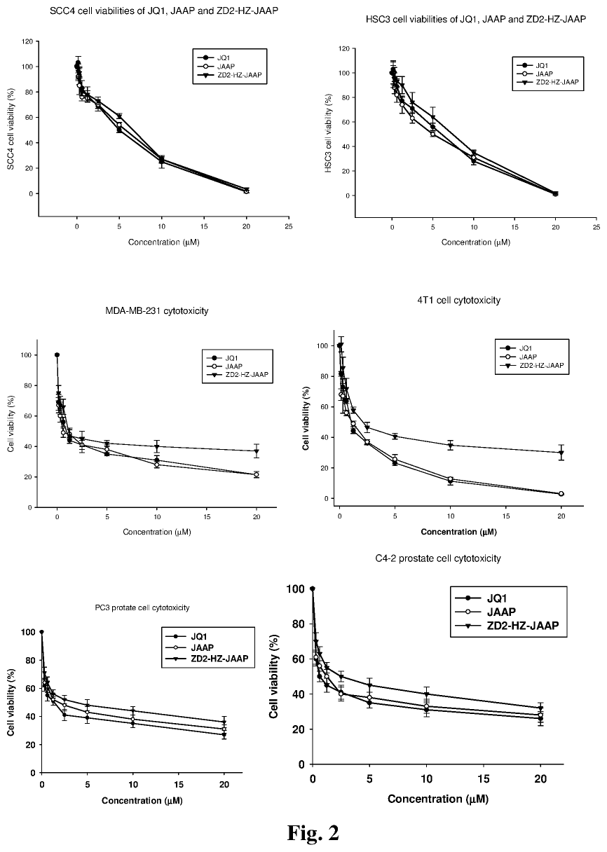

The cell cytotoxicity of ZD2-HZ-JAAP was tested using DLD-1-DR drug resistant colon cancer, PANC-1 and BXPC3 pancreatic cancer, CAL27, SCC4 and HSC3 head and neck cancer cells, MDA-MB-231 and 4T1 breast cancer cells, PC3 and C4-2 prostate cancer cells. After 48 hours incubation, the cytotoxicity of ZD2-HZ-JAAP is lower than that of JQ1 or JAAP due to partial release of JAAP by partial cleavage of the hydrazone bonds in cell culture condition. See FIG. 2.

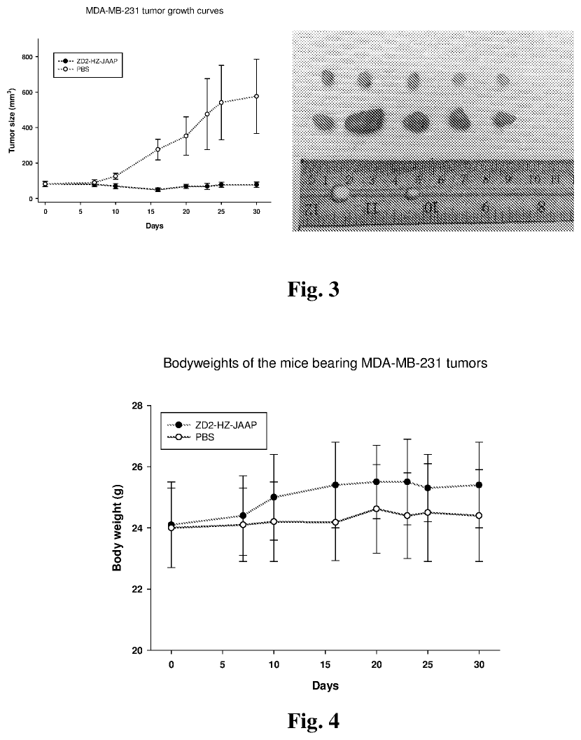

The in vivo anti-tumor activity of ZD2-HZ-JAAP was evaluated in the nude mice carrying human MDA-MB-231 triple negative breast cancer tumors. See FIG. 3. The ZD2-HZ-JAAP drug conjugate was administrated to the mice with human MDA-MB-231 tumors by daily i.p. injection at a dose of 4.4 μmol/kg/day for 30 days. The tumor sizes and body weights of the mice were measured twice a week. The tumor growth was also monitored using bioluminescence imaging on the day 1, 16, and 25 after drug administration, and MRI imaging on the day 7 and 28 after the drug administration. For the ZD2-HZ-JAAP treatment group the tumor growth was inhibited with average tumor size of 80 mm³ after 30-days of drug treatment, while for the no-treatment control group the tumors were kept growing with average tumor size of 580 mm³ after 30-days of no treatment. For the body-weight of the mice there is no significant difference between drug treatment group and no-treatment group. See FIG. 4.

In literature, the treatment dose of JQ1 is 50 mg/kg/day or 0.11 mmol/kg/day by i.p. injection to treat the mice with tumors. The dose of ZD2-HZ-JAAP administered by the inventors is 4.4 μmol/kg/day, which is 25-times lower than JQ1 dose. Even at low dose as 4.4 μmol/kg/day, ZD2-HZ-JAAP efficiently inhibited the triple-negative breast cancer tumor growth in mice. The body weight of treatment group kept growing during the drug treatment period, which demonstrated that ZD2-HZ-JAAP is not toxic or low toxic with efficient treatment. Since ZD2-HZ-JAAP is a target-drug that is delivered and accumulated to the tumor site, which results in low toxicity for normal tissues. Target-drug delivery results in high drug concentration at tumor site, and low concentration at normal tissues with efficient killing of tumor cells and low toxicity to normal cells. In conclusion, ZD2-HZ-JAAP efficiently inhibits the tumor growth at low drug dose with very low toxicity.

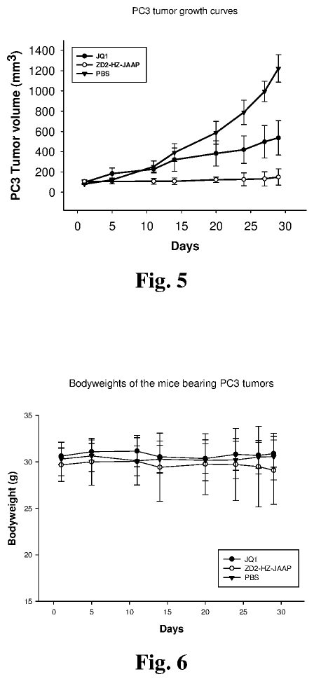

Anti-tumor efficacy of ZD2-HZ-JAAP conjugate was also investigated using the mice bearing PC3 prostate or 4T1 breast tumor xenografts. See FIG. 5. For tumor xenografts, PC3 prostate or 4T1 breast cancer cells suspended in Matrigel-PBS were subcutaneously injected into the flank of nude mice or mammary fat pads of BALB/C mice, respectively. Tumor volumes were monitored and measured twice a week using a Vernier caliper. When the average tumor volumes reached 100 mm³ for PC3 tumors, and 45 mm³ for 4T1 tumors, the mice bearing tumor xenografts were randomized into PBS control, JQ1 and ZD2-HZ-JAAP treatment groups (n=6 for PC3 tumors or 5 for 4T1 tumors). Free JQ1 and ZD2-HZ-JAAP conjugates were administrated by daily intraperitoneal injection for 29 days at a dose of 7.34 μmol/kg/day. Tumor size and bodyweight were monitored twice a week. The tumor sizes were measured with Vernier calipers, and tumor volumes were calculated using the equation V=(L*W2)/2.

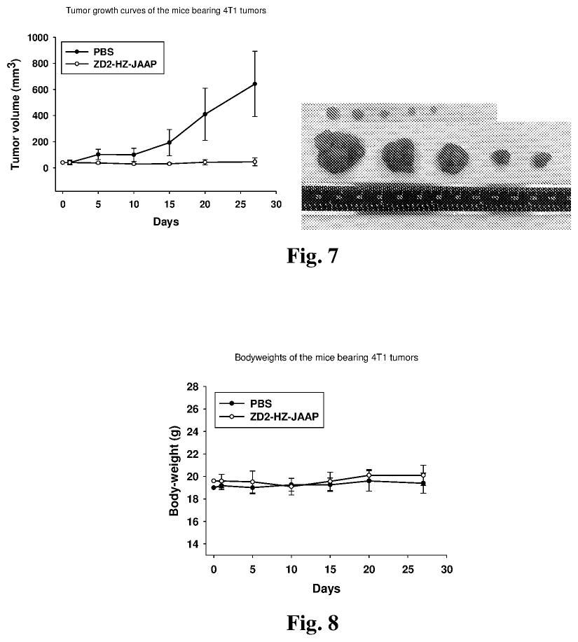

Treatment with ZD2-HZ-JAAP inhibited the tumor growth compared to JQ1 or PBS treatment control group. For PC3 tumors by the end of the treatment the tumor volume of ZD2-HZ-JAAP treated group (150±80 mm³, mean±SD) is significantly smaller than that of JQ1 (537±170 mm³) and PBS treated control group (1223±136 mm³). For 4T1 tumors by the end of the treatment the tumor volume of ZD2-HZ-JAAP treated group (46±30 mm³, mean±SD) is significantly smaller than that of PBS treated control group (645±240 mm³). All mice in ZD2-HZ-JAAP treatment group maintained bodyweight such that bodyweight change was within 10% of initial weight, and all mice were survived during the 29 days of treatment period. Free JQ1 treatment didn't efficiently suppress tumor growth with the same dose as used for ZD2-HZ-JAAP group. In conclusion, ZD2-HZ-JAAP efficiently inhibits tumor growth with low toxicity. See FIGS. 6-8.

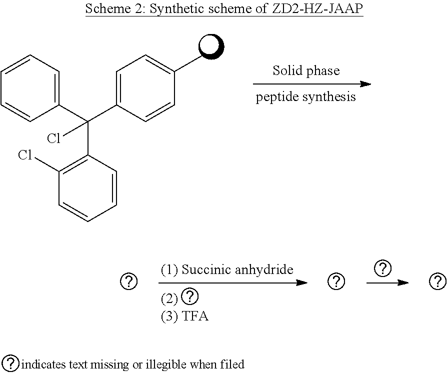 [SMILES: Cc1ccc(C(Cl)(c2ccccc2)c2ccccc2Cl)cc1]

3. Synthesis and Characterization of ZD2-AMs-JAAP Conjugated with Amide Bond without Linker

Synthesis of ZD2-AMs-JAAP MW-1132. The ZD2-Ams-JAAP MW-1132 was synthesized using standard solid-phase synthesis method. The ZD2 resin 300 mg mixed with JQ1-COOH 12 mg in the presence of an excess of in DMF and shaken for 4 hours, and then cleaved by TFA, yield 90% and purified by preparative HPLC. The molecular weight was determined by ESI mass spectrometry, m/z 1132 (calculated MW 1131.65). The ZD2-AMs-JAAP MW-1132 was analyzed by HPLC with a peak time 14.52 min and an UV spectrum peak at 258 nm.

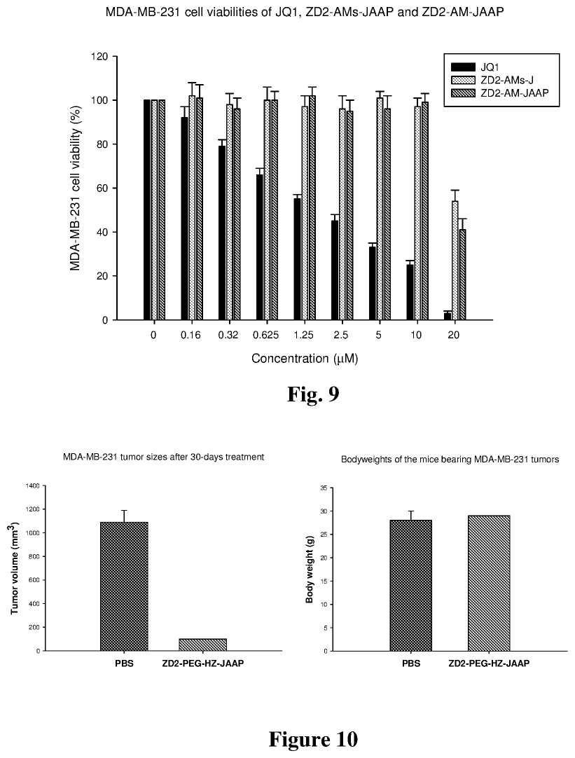

In vitro study demonstrated that the cell cytotoxicity of ZD2-AMs-JAAP MW-1132 is lower than that of JQ1 for MDA-MB-231 breast cancer cells. See FIG. 9.

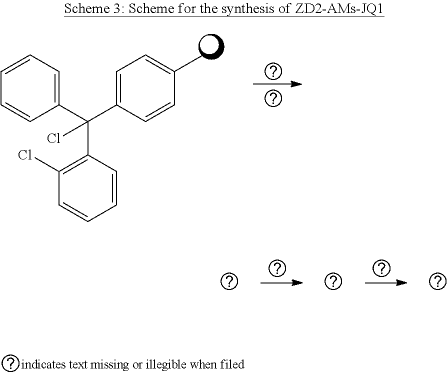 [SMILES: Cc1ccc(C(Cl)(c2ccccc2)c2ccccc2Cl)cc1]

Synthesis of ZD2-AM-JAAP with a Linker

The ZD2-AM-JAAP MW-1245 was synthesized using standard solid-phase synthesis method. The ZD2 resin 120 mg was mixed with Fmoc-6-amino-hexanoic acid 70 mg, an excess of HBTU and shaken for 3 hours, and washed with DMF for 3 times. JQ1-COOH (12 mg) was then added shaken for 4 hours, and cleaved from resin using TFA. Yield 90% and purified by preparative HPLC. The molecular weight was determined by ESI mass spectrometry, m/z 1245 (calculated MW 1244.81). The ZD2-AM-JAAP MW-1245 was analyzed by HPLC with a peak time 14 min and an UV spectrum peak at 257 nm.

In vitro study demonstrated that the cell cytotoxicity of ZD2-AM-JAAP MW-1245 is lower than that of JQ1 for MDA-MB-231 breast cancer cells. See FIG. 9.

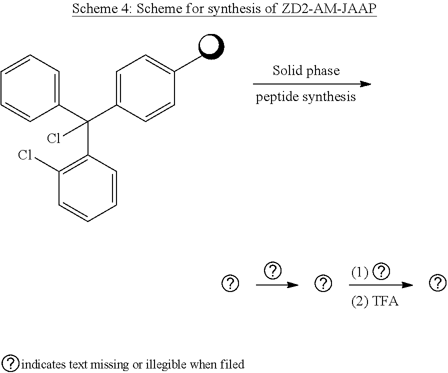 [SMILES: Cc1ccc(C(Cl)(c2ccccc2)c2ccccc2Cl)cc1]

### 4. Synthesis and Characterization of ZD2-PEG-HZ-JAAP Polymer Conjugate Via a Hydrazone Bond

The PEG-NHS ester was synthesized by mixing 20 KDa PEG-15A 1 g with DCC 100 mg, NHS 250 mg in DMF 14 ml, stirred at r.t. overnight, centrifuged to remove the precipitate DCU. The supernatant was precipitated in ether to obtain PEG-NHS active ester. The PEG-NHS ester 1 g was mixed with ZD2 peptide 200 mg, DIPEA 0.3 ml, DMF 15 ml, stirred at r.t. overnight, next day added t-butyl carbazate 250 mg, stirred overnight again, and then precipitated in ether to obtain the hydrazide ZD2-PEG-NHNH₂. The ZD2-PEG-NHNH₂ 910 mg was mixed with JAAP 100 mg, DMF 10 ml, and molecular sieve 400 mg, and stirred at r.t. overnight. After reaction, the mixture was centrifuged to remove the molecular sieve. The supernatant was precipitated in ether, and the precipitate was dried under vacuum. The obtained powder was dissolved in water, and solution filtrated through a 10 KDa cut-off filter. The filtrate was freeze-dried to obtain ZD2-PEG-HZ-JAAP.

The 20 KDa ZD2-PEG-HZ-JAAP conjugate was characterized by UV spectrometer. On the UV spectrum of the polymer, the special absorbance of the peak at 260 nm for JAAP was used to determine the JAAP contents. For the ZD2-PEG-HZ-JAAP polymer the JAAP content is 4 JAAP molecules per polymer chain.

The in vivo anti-tumor activity of ZD2-PEG-HZ-JAAP was evaluated in the mice carrying human MDA-MB-231 triple-negative breast cancer tumors. ZD2-PEG-HZ-JAAP was administrated to the mice by i.v. injection at a dose of 4 μmol/kg of JAAP content 3-injections/week for 30 days. The tumor sizes and body weights of the mice were measured twice a week. For the ZD2-PEG-HZ-JAAP treatment group, the tumor growth was inhibited with average tumor size of 110 mm³ for 30 days of drug treatment, while for the no-treatment control group the tumors were kept growing with average tumor size of 1008 mm³ for 30 days of no-treatment. For the body weight of the mice there is no significant difference between drug treatment group and no-treatment group. See FIG. 10.

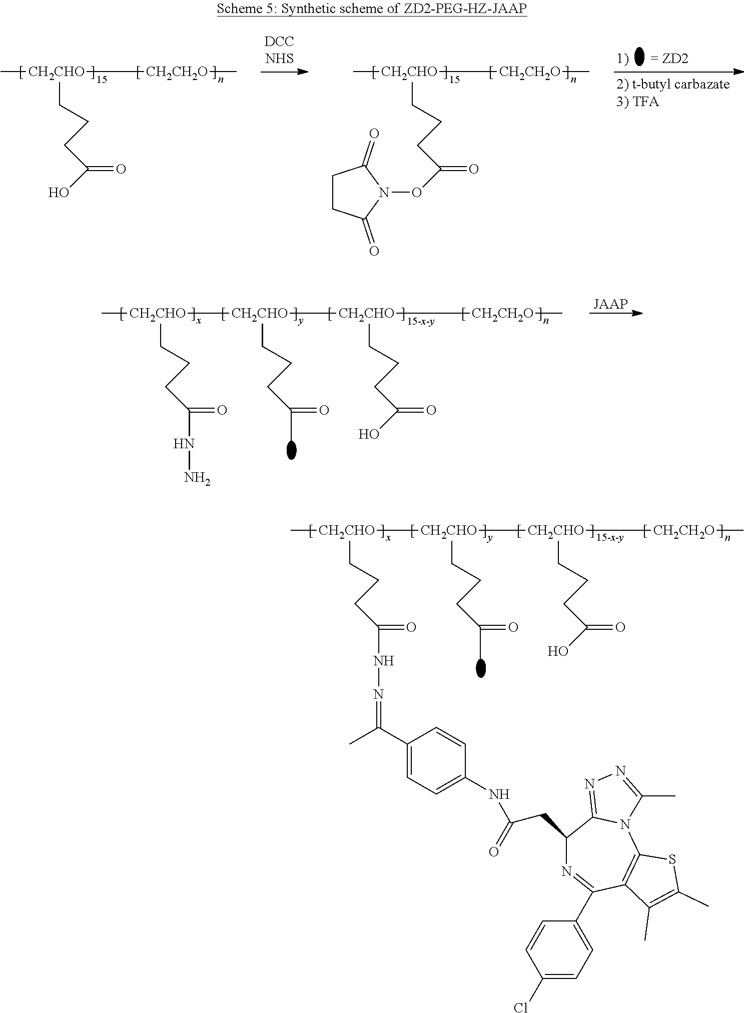

### Example 2: Data for ZD2 Doxorubicin Conjugates

Three types of the ZD2-doxorubicin (DOX) conjugates, including ZD2 conjugated with DOX via a hydrazone bond, ZD2 conjugated with DOX via amide bond, and PEG polymer conjugated with both ZD2 and DOX, were designed, synthesized, characterized, and evaluated in vitro and in vivo.

### 1, ZD2-Doxorubicin Conjugated Via a Hydrazone Bond ZD2-HZ-DOX

ZD2 on resin was synthesized using standard solid phase synthesis. ZD2 resin (1 g) was reacted with 500 mg succinic anhydride, and then 400 mg t-butyl carbazate and an excess of HBTU, and cleaved from resin using trifluoroacetic acid (TFA) to obtain ZD2-hydrazide (ZD2-NHNH₂, MW 863, MALDI m/z 863.78), 200 mg. For the preparation of the conjugate, 50 mg ZD2-hydrazide was mixed with 37 mg DOX, added molecular sieve 200 mg and DMF 3 ml, and stirred at r.t. overnight. The mixture was centrifuged and the supernatant was purified by preparative HPLC to obtain ZD2-DOX conjugate with Schiff base bond.

The ZD2-HZ-DOX conjugate with a hydrazone bond was confirmed by ESI (m/z 1388 and 1410 for M+Na), analyzed by HPLC with the peak showed at 16.7 min compared to doxorubicin at 18.7 min, and characterized by UV spectrum with an UV peak at 480 nm.

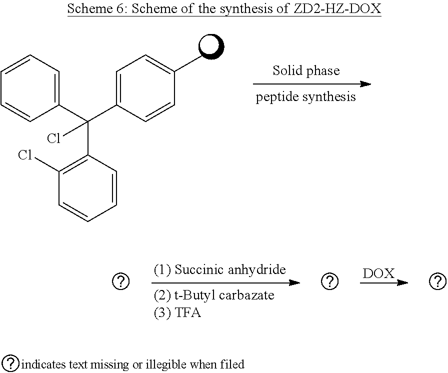

### 2, ZD2-Doxorubicin Conjugated Via an Amide Bond ZD2-AM-DOX

ZD2-AM-DOX conjugate on resin was synthesized using standard solid phase synthesis. ZD2 resin 1 g was reacted with succinic anhydride 500 mg, and then doxorubicin 200 mg and an excess of HBTU, and cleared from resin using trifluoroacetic acid (TFA) to obtain ZD2-AM-DOX conjugated via amide bond (MW 1374.38, MALDI m/z 1418.2 MW+2Na), ZD2-AM-DOX 260 mg. The mixture was purified by preparative HPLC to obtain ZD2-AM-DOX conjugate with an amide bond.

The ZD2-AM-DOX conjugate was confirmed by MALDI-TOF (m/z 1418.2 MW+2Na), analyzed by HPLC with the peak showed at 19.2 min, and characterized by UV spectrum with an UV peak at 475 nm.

The cell cytotoxicity of ZD2-AM-DOX conjugate is lower than that of doxorubicin when tested with PC3 prostate cancer, 4T1 and MDA-MB-231 breast cancer cells at the concentrations of 2.5, 5 and 10 μM. The reason for the lower cytotoxicity is the slow release of DOX from the ZD2-AM-DOX conjugated with an amide bond. See FIG. 11.

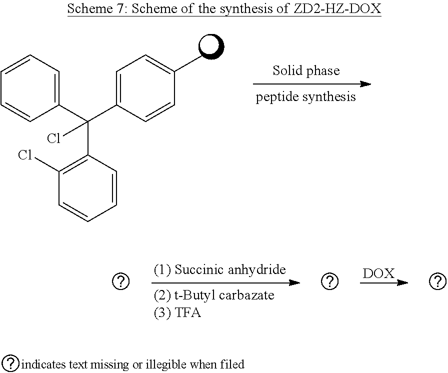

### 3, PEG Copolymer Conjugate of ZD2 and DOX Via a Hydrazone Bond, ZD2-PEG-HZ-DOX

The PEG-NHS ester was synthesized by mixing 20 KDa PEG-15A 1 g with DCC 100 mg, NHS 250 mg in DMF 14 ml, stirred at r.t. overnight, centrifuged to remove the precipitate DCU. The supernatant was precipitated in ether to obtain PEG-NHS active ester. The PEG-NHS ester 1 g was mixed with ZD2 peptide 200 mg, DIPEA 0.3 ml, DMF 15 ml, stirred at r.t. overnight. The next day 250 mg t-butyl carbazate was added, stirred overnight again, and then precipitated in ether to obtain the protected hydrazide ZD2-PEG-NHNH-Bu^{t}. Deprotection with TFA of the polymers gave ZD2-PEG-NHNH₂. ZD2-PEG-NHNH₂ (310 mg) was mixed with DOX 120 mg, DMF 10 ml, and molecular sieve 400 mg, and stirred at rt overnight. After reaction, the mixture was centrifuged to remove the molecular sieve. The supernatant was precipitated in ether, and the precipitate was dried under vacuum. The obtained powder was dissolved in water, and solution filtrated through a 10 KDa cut-off filter. The filtrate was freeze-dried to obtain ZD2-PEG-HZ-DOX.

The 20 KDa ZD2-PEG-HZ-DOX polymer was characterized by UV spectrometer. On the UV spectrum of the polymer, the special absorbance of the peak at 495 nm with DOX extinction coefficient 10000 M⁻¹ cm⁻¹ was used to determine the DOX contents. For the ZD2-PEG-HZ-DOX polymer conjugate, the DOX content is 8 DOX molecules per polymer chain.

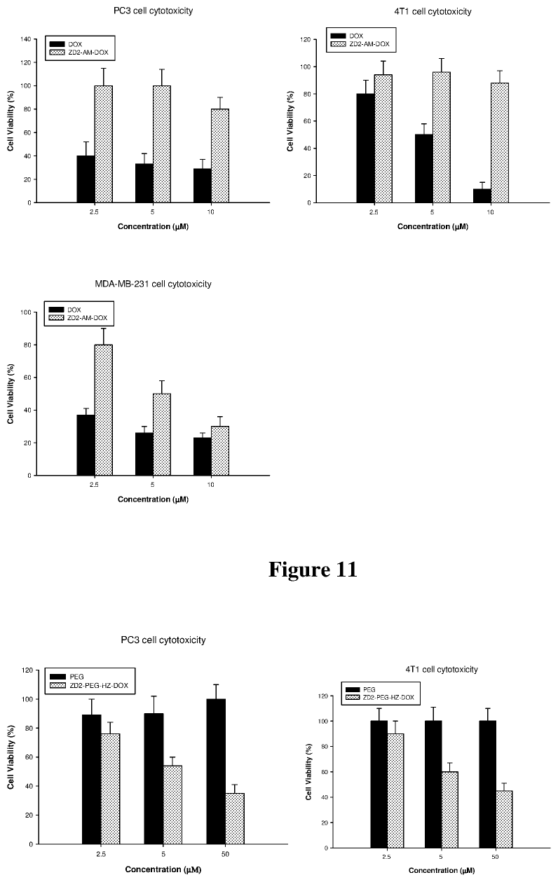

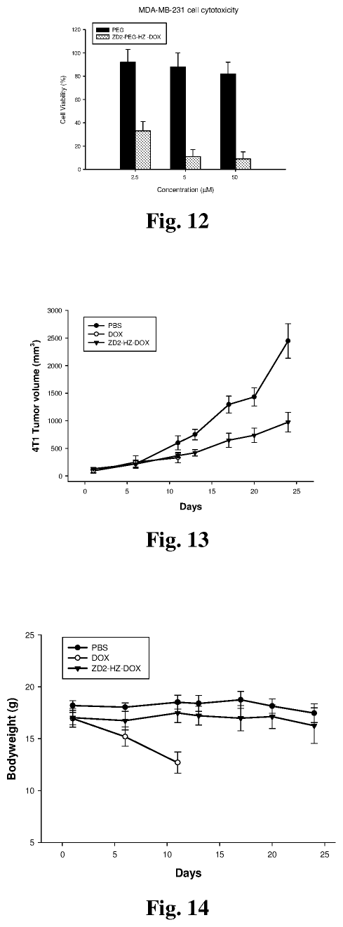

The cell cytotoxicity of the ZD2-PEG-HZ-DOX was tested using PC3 prostate cancer, 4T1 and MDA-MB-231 breast cancer cells at concentrations of 2.5. 5, and 50 μM, and demonstrated that the cytotoxicity of ZD2-PEG-HZ-DOX is higher than that of PEG due to the release of DOX from the PEG polymer conjugate by cleavage of the Schiff base bond. See FIG. 12.

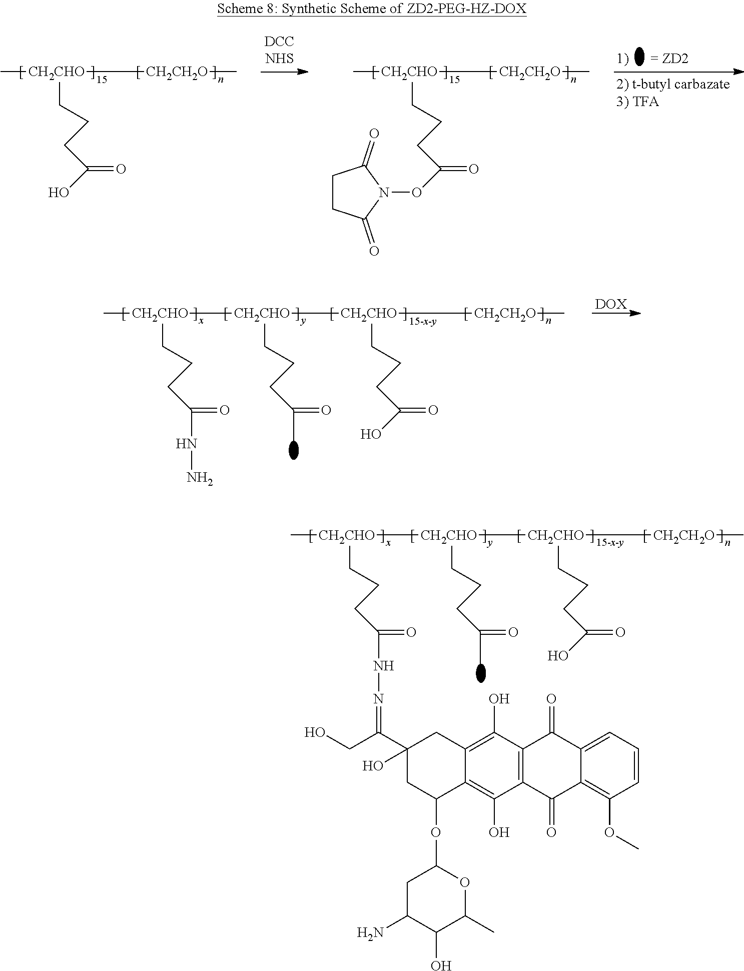

### 4. Anti-Tumor Efficacy of ZD2-HZ-DOX (MW 1388) Conjugate

Anti-tumor efficacy of ZD2-HZ-DOX conjugate was investigated using the mice bearing 4T1 breast tumor xenografts. For tumor xenografts, 2×10⁵ 4T1 breast cancer cells suspended in Matrigel-PBS were injected into the mammary fat pads of each BALB/C mouse. Tumor volumes were monitored and measured twice a week using a Vernier caliper. When the average tumor volumes reached 100 mm³, the BALB/C mice bearing 4T1 breast tumors were randomized into PBS control, free doxorubicin and ZD2-HZ-DOX treatment groups (n=5). Free doxorubicin and ZD2-HZ-DOX conjugates were administrated by intraperitoneal injection for 23 days at a dose of 8.28 μmol/kg/week with 3-injections per week, and 2.76 μmol/kg for each injection. Tumor size and bodyweight were monitored twice a week. The tumor sizes were measured with Vernier calipers, and tumor volumes were calculated using the equation V=(L*W2)/2.

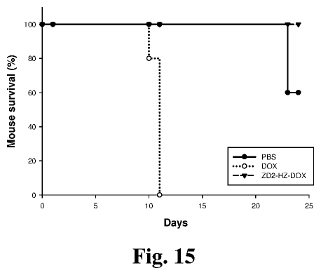

Treatment with ZD2-HZ-DOX inhibited the tumor growth compared to PBS treatment control group. By the end of the treatment the tumor volume of ZD2-HZ-DOX treated group (975±177 mm³, mean±SD) is significantly smaller than that of PBS treated control group (2447±311 mm³). All mice in the ZD2-DOX treatment group maintained a bodyweight within 10% of initial weight, and all mice survived during the 24-day treatment period. The mice in the free doxorubicin treatment group lost bodyweight quickly with loss of 25% of initial weight at day 10. One mouse died on day 10, and all remaining mice died on day 11 due to the toxicity of free doxorubicin. In PBS vehicle treatment group, two mice died on day 23 due to big tumor sizes. In conclusion, ZD2-HZ-DOX efficiently inhibits tumor growth with lower toxicity compared to free doxorubicin. See FIGS. 13-15.

The complete disclosure of all patents, patent applications, and publications, and electronically available materials cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An anticancer peptide conjugate comprising the following formula:
P-L-A wherein:
P is a peptide that includes an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or variants thereof in which one or more L-amino acids have been replace with a corresponding D-amino acid;
A is an antitumor agent; and
L is an optional linker that covalently links the peptide to the antitumor agent, and pharmaceutically acceptable salts thereof.

2. The anticancer peptide conjugate of claim 1, wherein the amino acid sequence is SEQ ID NO: 1.

3. The anticancer peptide conjugate of claim 1, wherein the linker is a non-peptide linker.

4. The anticancer peptide conjugate of claim 3, wherein the non-peptide linker is a non-peptide aliphatic or heteroaliphatic linker.

5. The anticancer peptide conjugate of claim 1, wherein the linker is covalently attached to the antitumor agent through an amide or hydrazone bond.

6. The anticancer peptide conjugate of claim 1, wherein the antitumor agent is a BET bromodomain inhibitor.

7. The anticancer peptide conjugate of claim 1, wherein the BET bromodomain inhibitor is JQ1 or JAAP.

8. The anticancer peptide conjugate of claim 1, wherein P is a peptide that includes an amino acid sequence selected from the group consisting of SEQ ID NO: 10, SEQ ID NO: 11, SEQ ID NO: 12, SEQ ID NO: 13, and SEQ ID NO: 14.

9. The anticancer peptide conjugate of claim 1, wherein the conjugate is selected from the group consisting of ZD2-HZ-JAAP, ZD2-AMs-JQ1, ZD2-AM-JAAP, ZD2-PEG-HZ-JAAP, ZD2-HZ-DOX, ZD2-PEG-HZ-DOX, and ZD2-AM-DOX.

10. A method of treating cancer in a subject, comprising administering a therapeutically effective amount of an anticancer peptide conjugate to a subject in need thereof, the anticancer peptide conjugate comprising the following formula:
P-L-A, wherein:
P is a peptide that includes an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or variants thereof in which one or more L-amino acids have been replace with a corresponding D-amino acid;
A is an antitumor agent; and
L is an optional linker that covalently links the peptide to the antitumor agent, and pharmaceutically acceptable salts thereof.

11. The method of claim 10, wherein the cancer is breast cancer, oral cancer, pancreatic cancer, or prostate cancer.

12. The method of claim 10, wherein the cancer is drug-resistant cancer.

13. The method of claim 10, wherein the anticancer peptide conjugate is administered together with a pharmaceutically acceptable carrier.

14. The method of claim 10, wherein the amino acid sequence of the anticancer peptide conjugate is SEQ ID NO: 1.

15. The method of claim 10, wherein the linker is a non-peptide linker.

16. The method of claim 15, wherein the non-peptide linker of the anticancer peptide conjugate is a non-peptide aliphatic or heteroaliphatic linker.

17. The method of claim 10, wherein the linker of the anticancer peptide conjugate is covalently attached to the antitumor agent through an amide or hydrazone bond.

18. The method of claim 10, wherein the antitumor agent of the anticancer peptide conjugate is a BET bromodomain inhibitor.

19. The method of claim 18, wherein the BET bromodomain inhibitor is JQ1 or JAAP.

20. The method of claim 10, wherein P of the anticancer peptide conjugate is a peptide that includes an amino acid sequence selected from the group consisting of SEQ ID NO: 10, SEQ ID NO: 11, SEQ ID NO: 12, SEQ ID NO: 13, and SEQ ID NO: 14.

21. The method of claim 10, wherein the anticancer peptide conjugate is selected from the group consisting of ZD2-HZ-JAAP, ZD2-AMs-JQ1, ZD2-AM-JAAP, ZD2-PEG-HZ-JAAP, ZD2-HZ-DOX, ZD2-PEG-HZ-DOX, and ZD2-AM-DOX.
